SEQUENCE LISTING (25 sequences)

SEQ ID NO: 1 - protein, 14 aa, Artificial Sequence
  Artificially synthesized peptide
LDPAKDCGDQKYAY

SEQ ID NO: 2 - protein, 14 aa, Artificial Sequence
  Artificially synthesized peptide
LDPSKDCGDPKYAY

SEQ ID NO: 3 - protein, 15 aa, Artificial Sequence
  Artificially synthesized peptide
AYFNGCSSPTAPLDP

SEQ ID NO: 4 - protein, 10 aa, Artificial Sequence
  Artificially synthesized peptide
DPSKDCGDPK

SEQ ID NO: 5 - protein, 11 aa, Artificial Sequence
  Artificially synthesized peptide
DPSKDCGDPKY

SEQ ID NO: 6 - protein, 12 aa, Artificial Sequence
  Artificially synthesized peptide
DPSKDCGDPKYA

SEQ ID NO: 7 - protein, 13 aa, Artificial Sequence
  Artificially synthesized peptide
DPSKDCGDPKYAY

SEQ ID NO: 8 - protein, 14 aa, Artificial Sequence
  Artificially synthesized peptide
DPSKDCGDPKYAYF

SEQ ID NO: 9 - protein, 15 aa, Artificial Sequence
  Artificially synthesized peptide
DPSKDCGDPKYAYFN

SEQ ID NO: 10 - protein, 16 aa, Artificial Sequence
  Artificially synthesized peptide
DPSKDCGDPKYAYFNG

SEQ ID NO: 11 - protein, 17 aa, Artificial Sequence
  Artificially synthesized peptide
DPSKDCGDPKYAYFNGC

SEQ ID NO: 12 - protein, 18 aa, Artificial Sequence
  Artificially synthesized peptide
DPSKDCGDPKYAYFNGCS

SEQ ID NO: 13 - protein, 19 aa, Artificial Sequence
  Artificially synthesized peptide
DPSKDCGDPKYAYFNGCSS

SEQ ID NO: 14 - protein, 20 aa, Artificial Sequence
  Artificially synthesized peptide
DPSKDCGDPKYAYFNGCSSP

SEQ ID NO: 15 - protein, 21 aa, Artificial Sequence
  Artificially synthesized peptide
DPSKDCGDPKYAYFNGCSSPT

SEQ ID NO: 16 - protein, 25 aa, Artificial Sequence
  Artificially synthesized peptide
YGRKKRRQRRRLDPAKDCGDQKYAY

SEQ ID NO: 17 - protein, 382 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDDLEI

SEQ ID NO: 18 - protein, 382 aa, Mus musculus
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVEMHLKQIEIKKFKYGIEEHGKVKMRGGLLRTYIISILFKSVFEVAFLLIQWYIYGFSLSAVYTCKRDPCPHQVDCFLSRPTEKTIFIIFMLVVSLVSLALNIIELFYVFFKGVKDRVKGRSDPYHATTGPLSPSKDCGSPKYAYFNGCSSPTAPLSPMSPPGYKLVTGDRNNSSCRNYNKQASEQNWANYSAEQNRMGQAGSTISNSHAQPFDFPDDSQNAKKVAAGHELQPLAIVDQRPSSRASSRASSRPRPDDLEI

SEQ ID NO: 19 - protein, 25 aa, Artificial Sequence
  Artificially synthesized peptide
QGPLGVKDRVKGRSDPYHATTGPLD

SEQ ID NO: 20 - protein, 25 aa, Artificial Sequence
  Artificially synthesized peptide
PSKDCGDPKYAYFNGCSSPTAPLDP

SEQ ID NO: 21 - protein, 25 aa, Artificial Sequence
  Artificially synthesized peptide
APLDPMDPPGYKLVTGDRNNSSCRN

SEQ ID NO: 22 - protein, 22 aa, Artificial Sequence
  Artificially synthesized peptide
LDPSKDCGDPKYAYFNGCSSPT

SEQ ID NO: 23 - protein, 11 aa, Artificial Sequence
  Artificially synthesized peptide
GVKDRVKGRSD

SEQ ID NO: 24 - protein, 22 aa, Artificial Sequence
  Artificially synthesized peptide
KDLDYPSDPFAYKTGNGCSPCS

SEQ ID NO: 25 - protein, 14 aa, Artificial Sequence
  Artificially synthesized peptide
CPLDDKYSKDGYPA

(19) United States
(12) Patent Application Publication
(10) Pub. No.: US 2022/0380408 A1
(43) Pub. Date: Dec. 1, 2022

(54) COMPOSITIONS AND METHODS FOR INHIBITING NEOINTIMAL FORMATION

(71) Applicant: University of Virginia Patent Foundation, Charlottesville, VA (US)

(72) Inventors: Scott R. Johnstone, Roanoke, VA (US); Brant E. Isakson, Charlottesville (US)

(73) Assignee: University of Virginia Patent Foundation, Charlottesville (US)

(21) Appl. No.: 17/630,106

(22) PCT Filed: Jul. 27, 2020
(86) PCT No.: PCT/US2020/043710

§ 371 Date: Jan. 25, 2022

Related U.S. Application Data
(60) Provisional application No. 62/878,980, filed on Jul. 26, 2019.

(51) Int. Cl. C07K 7/08; A61P 35/00; A61P 9/10

(52) U.S. Cl. CPC C07K 7/08; A61P 35/00; A61P 9/10

(57) ABSTRACT

Provided are peptides that based on the amino acid sequences LDPAKDCGDQKYAY (SEQ ID NO: 1) and LDPSKDCGDPKYAY (SEQ ID NO: 2), optionally wherein the peptide includes an N-terminal stearate modification. Also provided are method for using the disclosed peptides for inhibiting neointima formation in mammals, methods for inhibiting division and/or proliferation of vascular smooth muscle cells, and uses of the disclosed peptides for treating cardiovascular diseases and/or disorder associated with undesirable vascular SMC proliferation, which in some embodiments can re-late to inhibiting neointima formation in a mammal.

### CROSS-REFERENCE TO RELATED APPLICATION

This application claims the benefit of U.S. Provisional Patent Application Ser. No. 62/878,980, filed Jul. 26, 2019, the disclosure of which is incorporated herein by reference in its entirety.

### GRANT STATEMENT

This invention was made with government support under Grant Nos. HL120840 and HL137112 awarded by National Institutes of Health. The Government has certain rights in the invention.

### TECHNICAL FIELD

The presently disclosed subject matter relates to diagnostics and therapeutics. In particular, it relates to therapeutic compositions and methods for using the same to inhibit vascular smooth muscle cell (SMC) proliferation, which in some embodiments can be employed to disrupt neointimal formation in mammalian vascular tissues.

### BACKGROUND

Altered gap junction functions have been associated with pathological cell proliferation for 50 years (Loewenstein & Kanno, 1966; Chadjichristos et al., 2006; Liao et al., 2007; Aasen et al., 2017). Critically, the biological determinants and mechanistic controls of this relationship are not known, and these unsolved questions are a central tenet of this application. One striking example of gap junction-associated proliferation occurs in the smooth muscle cells (SMC) of blood vessels that have been surgically stented due to blockage of the lumen (Chadjichristos et al., 2006; Hung et al., 2012; Zhang et al., 2016; Sun et al., 2017), a procedure performed over 500,000 times per year in the United States of America (Benjamin et al., 2017). Stenting causes undesirable SMC proliferation, which can result in neointimal formation via SMC proliferation from the walls of the blood vessel surrounding the stent. This blocks the artery and reduces blood flow leading to life-threatening complications.

Inhibiting SMC proliferation, while maintaining endothelial cell health, would be advantageous in reducing neointimal formation. Current therapeutics target reductions in cellular proliferation to lessen the burden of disease, but are ineffective as they also kill healthy endothelial cells. With respect to stents, therapeutic approaches to inhibit SMC proliferation utilize drug-eluting stents, providing short-term improvements in vessel function (Benedetto et al., 2018). However, the drugs used are non-specific, halting cellular proliferation not only in SMCs, but also in endothelial cells (ECs), where proliferation is required for the blood vessel to return to a functional state. As a result, more than 25% of stents fail within 5 years and require revascularization surgery (Parasca et al., 2016), leaving drug eluting stents no more efficacious than stents without drugs (Benedetto et al., 2016; Bonaa et al., 2016; Escaned et al., 2016; Benedetto et al., 2018).

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to peptide comprising, consisting essentially of, or consisting of the amino acid sequence LDPAKDCGDQKYAY (SEQ ID NO: 1) or LDPSKDCGDPKYAY (SEQ ID NO: 2). In some embodiments, the peptide comprises an N-terminal stearate modification.

In some embodiments, the presently disclosed subject matter also relates to methods for inhibiting neointima formation in mammals, optionally humans. In some embodiments, the methods comprise contacting a vascular smooth muscle cell (SMC) with an effective amount of a composition comprising, consisting essentially of, or consisting of one or more peptides as disclosed herein. In some embodiments, the vascular SMC is present in a subject. In some embodiments, the vascular SMC is present in a blood vessel in a subject.

In some embodiments, the effective amount of the one or more peptides are present within and/or coated onto a stent and is formulated to release from the stent when in contact with a vascular SMC. In some embodiments, the one or more peptides are formulated to release from the stent over a course of time selected from the group consisting of one or more minutes, one or more hours, one or more days, one or more weeks, and one or more months.

In some embodiments, the presently disclosed subject matter also relates to methods for inhibiting division and/or proliferation of vascular SMCs. In some embodiments, the methods comprise contacting one or more vascular SMCs with an effective amount of a composition as disclosed herein. In some embodiments, the composition comprises, consists essentially of, or consists of one or more peptides as disclosed herein. In some embodiments, the one or more vascular SMCs are present in a subject. In some embodiments, the one or more vascular SMCs are present in a blood vessel in a subject. In some embodiments, the effective amount of the composition comprising, consisting essentially of, or consisting of one or more peptides as disclosed herein is present within and/or coated onto a stent. In some embodiments, the composition is formulated to release the one or more peptides from the stent over a course of time selected from the group consisting of one or more minutes, one or more hours, one or more days, one or more weeks, and one or more months.

In some embodiments, the presently disclosed subject matter also relates to uses of the presently disclosed peptides for treating a cardiovascular disease or disorder associated with undesirable vascular SMC proliferation, optionally wherein the undesirable vascular SMC proliferation is characterized by and/or results at least in part from neointima formation.

In some embodiments, the presently disclosed subject matter also relates to uses of the presently disclosed peptides for inhibiting neointima formation in a mammal.

In some embodiments, the presently disclosed subject matter also relates to peptides for use in treating cardiovascular diseases, disorders, or conditions associated with undesirable vascular SMC proliferation and/or inhibiting neointima formation. In some embodiments, the peptides comprise, consist essentially of, or consist of the amino acid sequence LDPAKDCGDQKYAY (SEQ ID NO: 1), the amino acid sequence

LDPSKDCGDPKYAY (SEQ ID NO: 2), or a combination thereof. In some embodiments, one or more of the peptides comprise an N-terminal stearate modification.

In some embodiments, the presently disclosed subject matter also relates to methods for identifying inhibitors of cyclin E/connexin43 interactions. In some embodiments, the methods comprise (a) combining a cyclin E polypeptide and a connexin43 polypeptide in the presence of one or more of the peptides disclosed herein under conditions sufficient to induce formation of a complex between the cyclin E polypeptide and the connexin43 polypeptide; and (b) determining an extent of binding of the one or more of the peptides disclosed herein to the complex in the presence and the absence of a candidate inhibitor of a cyclin E/connexin43 interaction, wherein a reduction in biding of the peptide of claim 1, the peptide or claim 2, or a combination thereof to the complex in the presence of the candidate inhibitor of a cyclin E/connexin43 interaction as compared to in the absence of the candidate inhibitor of a cyclin E/connexin43 interaction is indicative of the candidate inhibitor of a cyclin E/connexin43 interaction being an inhibitor of a cyclin E/connexin43 interaction. In some embodiments, the determining step comprises employing a Förster resonance energy transfer (FRET) fluorescence based method. In some embodiments, the candidate inhibitor of a cyclin E/connexin43 interaction is a small molecule or an antibody or an antigen-binding fragment thereof.

In some embodiments, the presently disclosed subject matter also relates to uses of the presently disclosed peptides for identifying inhibitors of cyclin E/connexin43 interactions. In some embodiments, one or more of the peptides as disclosed herein are employed in a competition assay with one or more candidate inhibitors of a cyclin E/connexin43 interaction. In some embodiments, the identifying employs a Förster resonance energy transfer (FRET) based method. In some embodiments, the one or more candidate inhibitors of the cyclin E/connexin43 interaction are small molecules or antibodies or antigen-binding fragments thereof.

Thus, it is an object of the presently disclosed subject matter to provide compositions and methods for modulating cyclin E/connexin43 interactions to modulate cyclin E/connexin43 biological activities.

This and other objects are achieved in whole or in part by the presently disclosed subject matter. Further, an object of the presently disclosed subject matter having been stated above, other objects and advantages of the presently disclosed subject matter will become apparent to those skilled in the art after consideration of the following Description, Figures, and EXAMPLES.

### BRIEF DESCRIPTION OF THE FIGURES

FIGS. 1A-1C provide an exemplary approach to CycliCx Peptide Discovery.

FIG. 1 is a depiction of a possible interaction between the C-terminus of the mouse connexin43 (Cx43) polypeptide (Cx43-CT) and a cyclin E polypeptide (top panel) and a schematic depiction of an exemplary arrangement for printing peptide sequences on nitrocellulose, with overlapping sequences derived from the full length Cx-CT amino acid sequence. FIG. 1B is an exemplary result from probing a nitrocellulose array containing phosphor-mimetic (pS, D) and phosphor-null versions of peptides 1 (QGPLGVKDRVKGRSDPYHATTGPLD; SEQ ID NO: 19), 2 (PSKDCGDPKYAYFNGCSSPTAPLDP; SEQ ID NO: 20), and 3 (APLDPMDPPGYKLVTGDRNNSSCRN; SEQ ID NO: 21) with a cyclin E protein followed by detection of peptide/cyclin E interactions with an anti-cyclin antibody. The lower panel of FIG. 1B provides a series of bar graphs that show the relative signal strengths of each peptide. FIG. 1C provides a summary of the regions of the mammalian connexin43 polypeptide (Cx43) from which the tested peptides were derived. The peptides tested correspond to the amino acid sequences set forth in Table 1.

TABLE 1
Peptide Sequences of Human Connexin43 Tested

| Peptide Sequence | SEQ ID NO: |
|---|---|
| DPSKDCGDPK | 4 |
| DPSKDCGDPKY | 5 |
| DPSKDCGDPKYA | 6 |
| DPSKDCGDPKYAY | 7 |
| DPSKDCGDPKYAYF | 8 |
| DPSKDCGDPKYAYFN | 9 |
| DPSKDCGDPKYAYFNG | 10 |
| DPSKDCGDPKYAYFNGC | 11 |
| DPSKDCGDPKYAYFNGCS | 12 |
| DPSKDCGDPKYAYFNGCSS | 13 |
| DPSKDCGDPKYAYFNGCSSP | 14 |
| DPSKDCGDPKYAYFNGCSSPT | 15 |

FIGS. 2A-2C provide the results of protein interaction studies in human coronary artery smooth muscle cells. FIG. 2A is a blot of immunoprecipitation experiments showing that the ability to bind with, and therefore activate, cyclin D was inhibited in the presence of the agent PGPC, which causes protein kinase C (PKC) phosphorylation of Cx43.

FIG. 2B is a bar graph showing that alterations in phosphorylation correspond with increased proliferation for cells treated with agents (PDGF and POVPC) that promote MAP kinase phosphorylation of connexin43 (Cx43) and decreased proliferation for PGPC-treated cells as demonstrated by flow cytometry of EDU incorporation. FIG. 2C is a proposed model whereby Cx43 phosphorylation acts as a molecular switch that controls cellular proliferation via MAPK phosphorylation of Cx43 or quiescence via PKC phosphorylation of Cx43.

FIGS. 3A-3F show the results of CycliCx peptide treatment of human coronary artery smooth muscle cells (hCASMCs). FIG. 3A shows FACS data for EDU incorporation (indicative of proliferation) of either untreated hCASMCs (no treatment; NT) or hCASMCs treated with by comparison PDGF, CxPepl (LDPSKDCGDPKYAYFNGCSSPT; SEQ ID NO: 22), or CxPep2 (GVKDRVKGRSD; SEQ ID NO: 23). FIG. 3B is a bar graph that summarizes the results of hCASMCs treated PDGF and also with one or more of a scrambled, negative control peptide (SCR1; KDLDYPSDPFAYKTGNGCSPCS; SEQ ID NO: 24), CxPepl

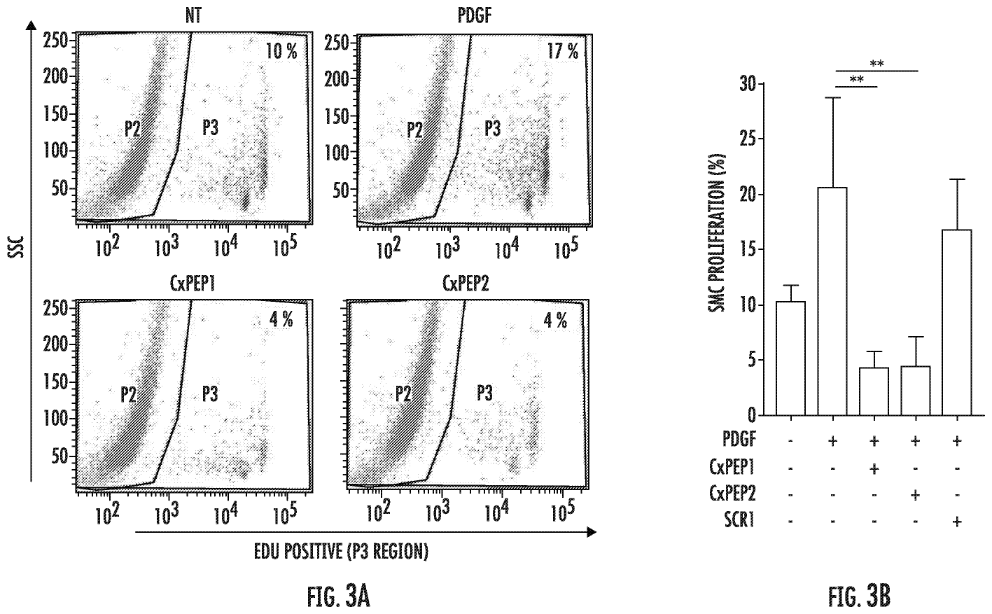

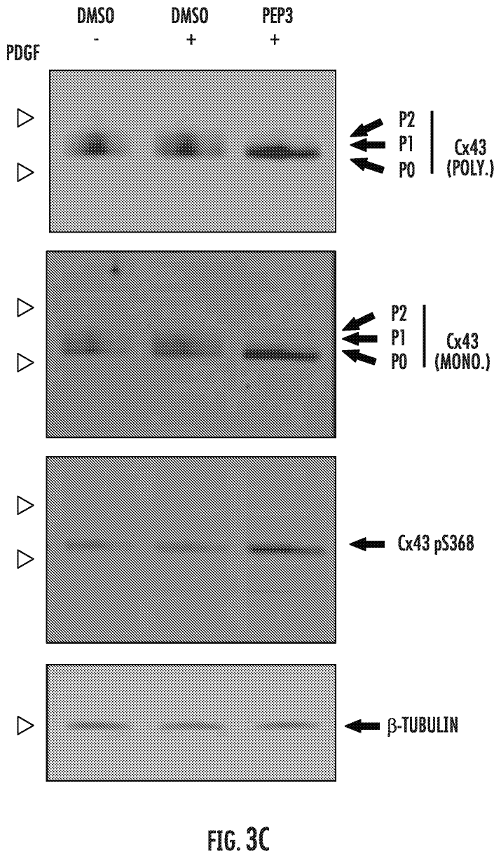

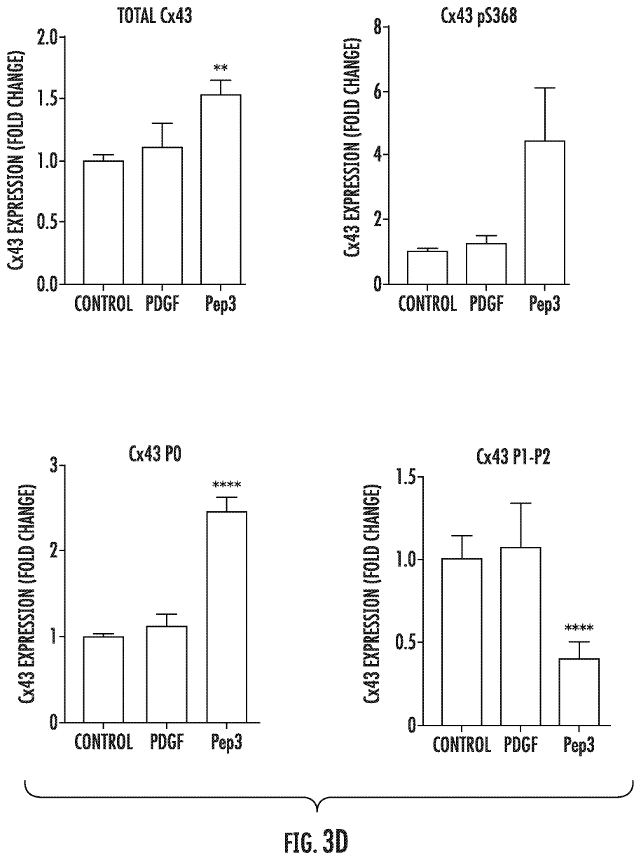

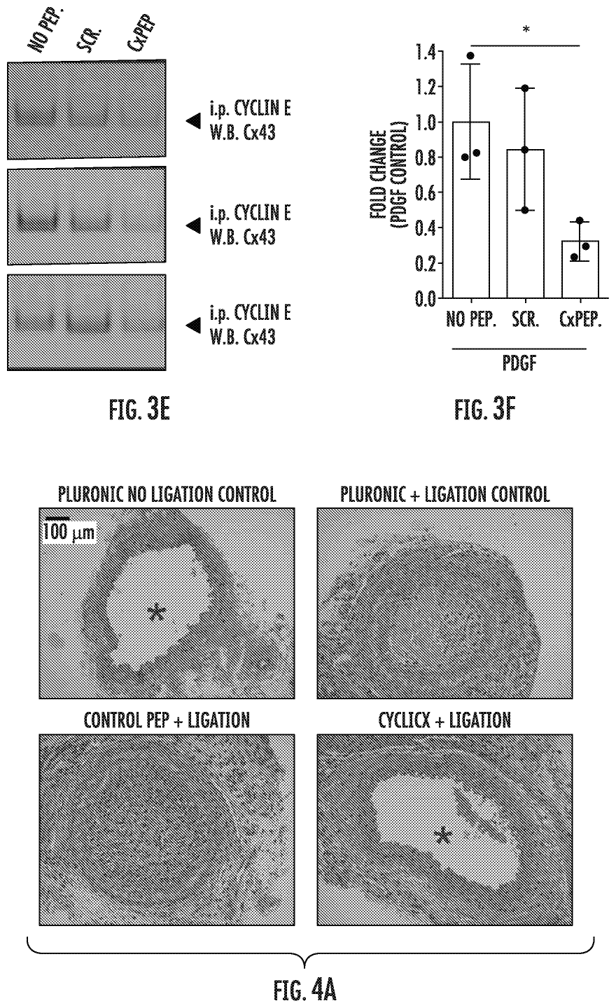

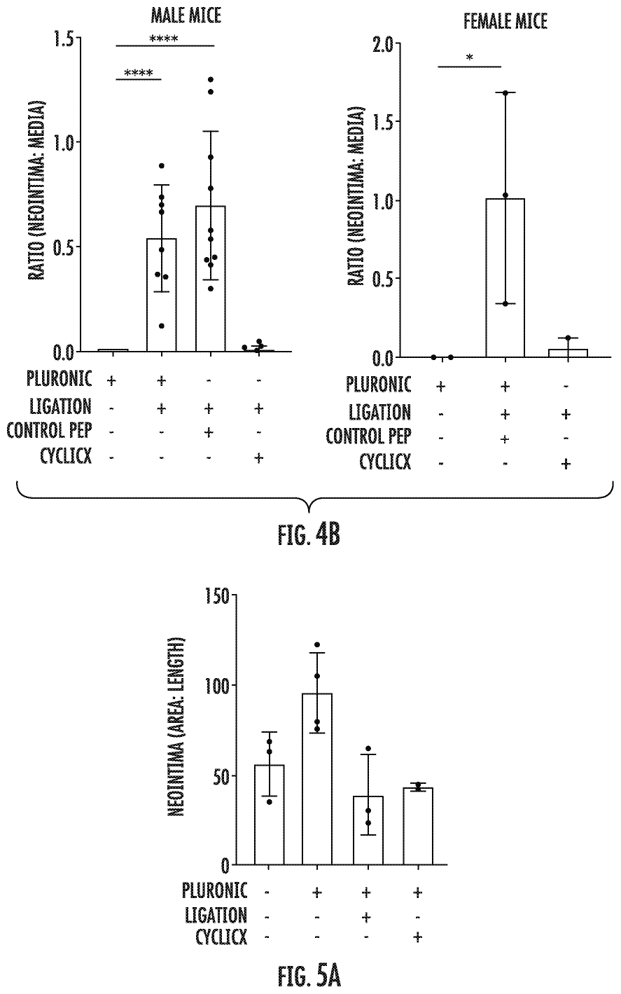

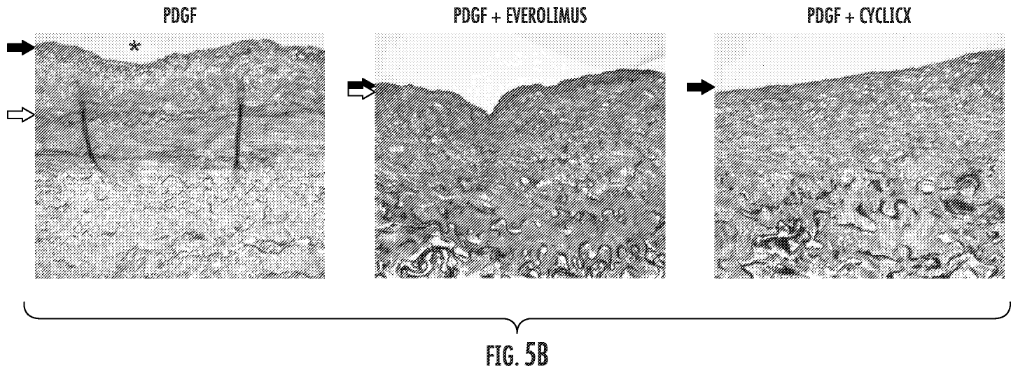

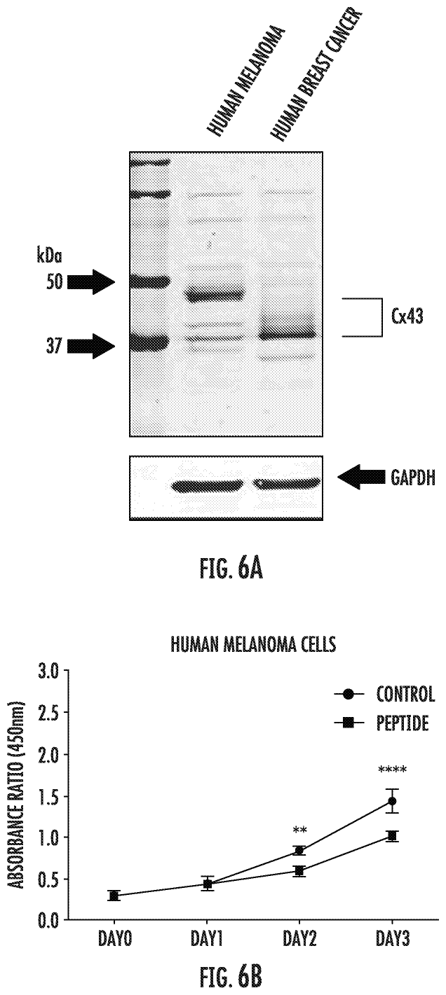

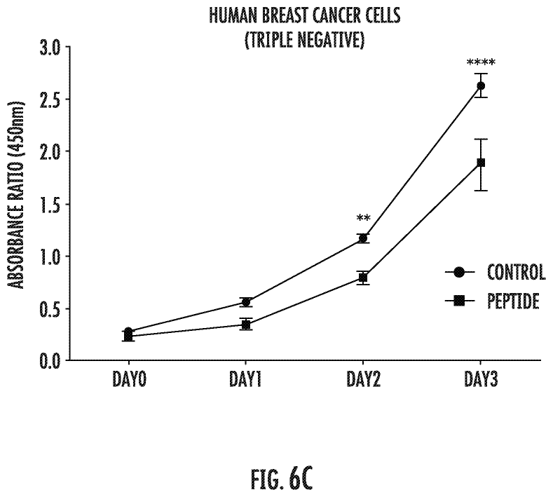

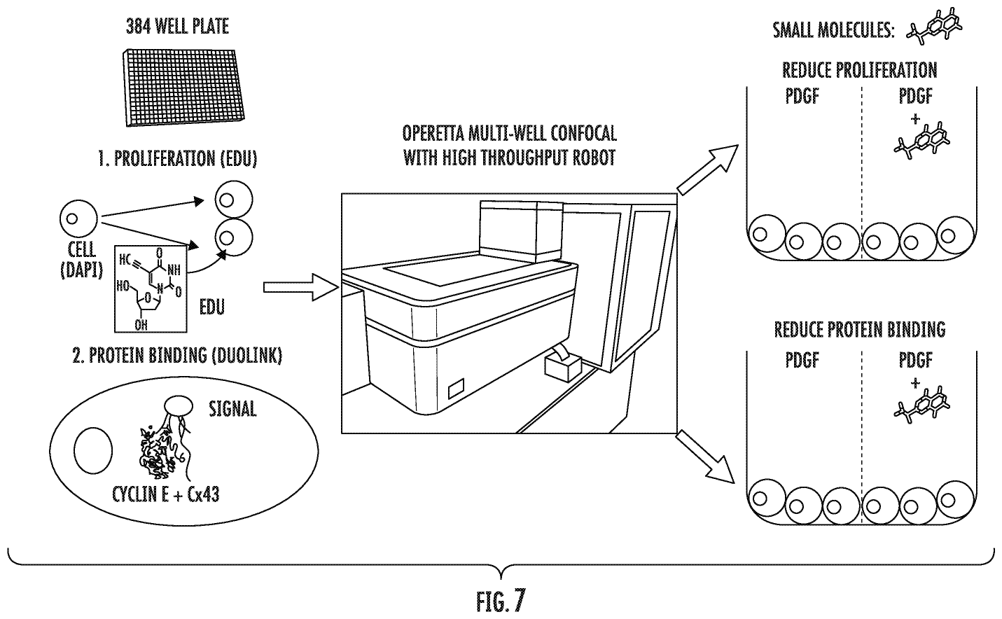

(LDPSKDCGDPKYAYFNGCSSPT; SEQ ID NO: 22), or CxPep2 (GVKDRVKGRSD; SEQ ID NO: 23). For each bar, “+” indicates that the treatment was present and “—” indicates that the treatment was absent. ** indicates p<0.01. Error bars represent ±SEM. FIG. 3C is a western blot showing that treatment with CycliCx (pep3(mouse); LDPSKDCGDPKYAY; SEQ ID NO: 2), altered the phosphorylation status of Cx43, where P0/P1/P2 in blots identifies differential phosphorylation states of Cx43 indicated with monoclonal (mono) and polyclonal (poly) antibodies for Cx43. In FIG. 3C, the Cx43 pS368 antibody identifies Cx43 phosphorylated by PKC the Serine 368 residue. β-tubulin is included as a loading control. FIG. 3D is a series of bar graphs presenting the data in FIG. 3C in an alternative form. **p<0.01; ****p<0.0001. Error bars represent ±SEM.

FIGS. 3E and 3F are a western blow and a bar graph, respectively, of co-immunoprecipitation studies in human SMC demonstrated that the CycliCx peptide (CxPep) significantly reduced interactions with cyclin E. No pep.: untreated human SMCs. Scr.: human SMCs treated with a control scrambled peptide (CPLDDKYSKDGYPA; SEQ ID NO: 26), CxPep: human SMCs treated with LDPSKDCGDPKYAY (SEQ ID NO: 2). *p<0.05. Error bars represent ±SEM.

FIGS. 4A and 4B show the results of peptide treatment of mouse carotid arteries. FIG. 4A is a series of micrographs of H&E stained mouse carotid artery sections showing the extent of neointimal formation induced by suture ligation. As shown, ligation produced significant neointimal formation, which was prevented by treatment with an exemplary

CycliCx peptide: LDPAKDCGDQKYAY (SEQ ID NO: 1) and not by the control peptide: AYFNGCSSPTAPLDP (SEQ ID NO: 3). FIG. 4B is two bar graphs showing that there was no obvious sex difference in the treatment of male or female mice. For each bar, “+” indicates that the treatment was present and “—” indicates that the treatment was absent. * indicates p<0.05, and **** indicates p<0.0001. Error bars represent ±SEM.

FIGS. 5A and 5B show the results of peptide treatment of human saphenous vein tissues. FIG. 5A is a bar graph showing the results of treatment of human saphenous vein tissues with PDGF and either everolimus or an exemplary CycliCx peptide

LDPAKDCGDQKYAY (SEQ ID NO: 1). For each bar, “+” indicates that the treatment was present and “—” indicates that the treatment was absent. * indicates p<0.05. Error bars represent ±SEM. Exemplary H&E stained an saphenous vein tissue sections showing the extent of neointimal formation induced is shown in FIG. 5B (White arrows indicate the internal elastic lamina, black arrows indicate the top of the formed neoinitma.

FIGS. 6A-6C show the results of treatment of human melanoma (A375-MA2) and triple negative breast cancer (Hs578T) cell lines with an exemplary CycliCx peptide (LDPAKDCGDQKYAY; SEQ ID NO: 1). FIG. 6A is a western blot showing that the pattern of Cx43 phosphorylation states in these untreated cell lines. FIGS. 6B and 6C are graphs showing that a single treatment of human melanoma (A375-MA2; FIG. 6B) or triple negative breast cancer (Hs578T; FIG. 6C) cells with the exemplary CycliCx peptide (30 μM; LDPAKDCGDQKYAY; SEQ ID NO: 1) at day 0 was sufficient to significantly inhibit proliferation in both cell types. Control: non-targeting peptide AYFNGCSSPTAPLDP (SEQ ID NO: 3); closed circles. Peptide: CycliCx peptide (30 μM; LDPAKDCGDQKYAY; SEQ ID NO: 1); closed squares. **p<0.01. ****p<0.0001. Error bars represent ±SEM.

FIG. 7 provides an exemplary high-throughput analysis methodology for small molecule inhibitors in cell proliferation and protein interaction. Smooth muscle cells plated in 384 well plates are tested for EDU (proliferation) and DuoLink signals (cyclin E/Cx43 interaction) on an Operetta system (pictured, middle) in the presence of small molecule inhibitors.

### DETAILED DESCRIPTION

### I. Definitions

In describing and claiming the presently disclosed subject matter, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. For example, in some embodiments, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

As used herein, the phrase “biological sample” refers to a sample isolated from a subject (e.g., a biopsy, blood, serum, etc.) or from a cell or tissue from a subject (e.g., RNA and/or DNA and/or a protein or polypeptide isolated therefrom). Biological samples can be of any biological tissue or fluid or cells from any organism as well as cells cultured in vitro, such as cell lines and tissue culture cells. Frequently the sample will be a “clinical sample” which is a sample derived from a subject (i.e., a subject undergoing a diagnostic procedure and/or a treatment). Typical clinical samples include, but are not limited to cerebrospinal fluid, serum, plasma, blood, saliva, skin, muscle, olfactory tissue, lacrimal fluid, synovial fluid, nail tissue, hair, feces, urine, a tissue or cell type, and combinations thereof, tissue or fine needle biopsy samples, and cells therefrom. Biological samples can also include sections of tissues, such as frozen sections or formalin fixed sections taken for histological purposes.

As used herein, term “comprising”, which is synonymous with “including,” “containing”, or “characterized by”, is inclusive or open-ended and does not exclude additional, unrecited elements and/or method steps. “Comprising” is a term of art used in claim language which means that the named elements are present, but other elements can be added and still form a composition or method within the scope of the presently disclosed subject matter. By way of example and not limitation, a pharmaceutical composition comprising a particular active agent and a pharmaceutically acceptable carrier can also contain other components including, but not limited to other active agents, other carriers and excipients, and any other molecule that might be appropriate for inclusion in the pharmaceutical composition without any limitation.

As used herein, the phrase “consisting of excludes any element, step, or ingredient that is not particularly recited in the claim. When the phrase “consists of appears in a clause of the body of a claim, rather than immediately following the preamble, it limits only the element set forth in that clause; other elements are not excluded from the claim as a whole. By way of example and not limitation, a pharmaceutical composition consisting of an active agent and a pharmaceutically acceptable carrier contains no other components besides the particular active agent and the pharmaceutically acceptable carrier. It is understood that any molecule that is below a reasonable level of detection is considered to be absent.

As used herein, the phrase “consisting essentially of limits the scope of a claim to the specified materials or steps, plus those that do not materially affect the basic and novel characteristic(s) of the claimed subject matter. By way of example and not limitation, a pharmaceutical composition consisting essentially of an active agent and a pharmaceutically acceptable carrier contains active agent and the pharmaceutically acceptable carrier, but can also include any additional elements that might be present but that do not materially affect the biological functions of the composition in vitro or in vivo.

With respect to the terms “comprising”, “consisting essentially of′, and “consisting of′, where one of these three terms is used herein, the presently disclosed and claimed subject matter encompasses the use of either of the other two terms. For example, “comprising” is a transitional term that is broader than both “consisting essentially of and “consisting of”, and thus the term “comprising” implicitly encompasses both “consisting essentially of and” consisting of′. Likewise, the transitional phrase “consisting essentially of is broader than” consisting of′, and thus the phrase “consisting essentially of implicitly encompasses” consisting of′.

As used herein, the terms “condition”, “disease condition”, “disease”, “disease state”, and “disorder” refer to physiological states in which diseased cells or cells of interest can be targeted with the compositions of the presently disclosed subject matter. In some embodiments, a disease is a cardiovascular disease, which in some embodiments is a cardiovascular disease, disorder, or condition associated with undesirable vascular smooth muscle cell proliferation, which in some embodiments can result in neointima formation.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Ayes (birds), and Mammalia (mammals)), and all Orders and Families encompassed therein. In some embodiments, a subject is a human.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter can exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ, complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce

F(ab′)₂ a dimer of Fab which itself is a light chain joined to V_{H} -C_{H1} by a disulfide bond. The F(ab′)₂ can be reduced under mild conditions to break the disulfide linkage in the hinge region, thereby converting the F(ab′)₂ dimer into an Fabi monomer. The Fabi monomer is essentially a Fab with part of the hinge region. While various antibody fragments are defined in terms of the digestion of an intact antibody, one of skill will appreciate that such fragments can be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all intact antibody molecules.

An “antibody light chain”, as used herein, refers to the smaller of the two types of polypeptide chains present in all intact antibody molecules.

The term “single chain antibody” refers to an antibody wherein the genetic information encoding the functional fragments of the antibody are located in a single contiguous length of DNA. For a thorough description of single chain antibodies, see U.S. Pat. Nos. 4,704,692; 4,946,778; and 5,395,750; each of which is incorporated herein by reference in its entirety.

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response can involve either antibody production, or the activation of specific immunologically-competent cells, or both. An antigen can be derived from organisms, subunits of proteins/antigens, killed or inactivated whole cells or lysates.

### II. Representative Compositions

Disclosed herein are therapeutic peptides, which in some embodiments are referred to herein as “CycliCx”, that have clear indications for the inhibition of vascular smooth muscle cell (SMC) division (proliferation), which is the key target in the treatment of cardiovascular diseases. The CycliCx peptides are characterized in some embodiments as having the ability to disrupt SMC proliferation and neointimal formation in mammalian (including but not limited to human and mouse) vascular tissues and blood vessels. The peptides of the presently disclosed subject matter are in some embodiments mimetics of the subsequences of the C-terminus of Cx43 (in some embodiments, amino acids 254-267 of a human Cx43 gene product as set forth in Accession No. NP 000156.1 (SEQ ID NO: 17) of the GENBANK® biosequence database or amino acids 254-267 of the mouse Cx43 gene produce as set forth in Accession No. NP_034418.1 (SEQ ID NO: 18) of the GENBANK® biosequence database). An exemplary 14 amino acid CycliCx peptide as described herein has been modified from the human or mouse Cx43 gene product to substitute two aspartate (D) mutations for serines 255 and 262 of Accession No. NP_000156.1 of the GENBANK® biosequence database (SEQ ID NO: 17). In some embodiments, a stearate-linker can be attached to the peptide to aid cellular permeability, optionally at the N-terminus. As shown above in Table 1, these peptides are based on the mouse Cx43 polypeptide sequence, which contains a serine at residue 257 (which is an alanine in the human sequence) and a proline at position 263 (which is a glutamine in the human sequence). Peptide 3 maintained the mouse residues at position 257 and 263, and these amino acids were substituted in the humanized peptide for alanine and glutamine, respectively.

In some embodiments, an exemplary CycliCx peptide comprises, consists essentially of, or consists of 14 amino acids, with a sequence of: stearate-LDPAKDCGDQKYAY (SEQ ID NO: 1), with exemplary aspartate mutations underlined.

The peptides of the presently disclosed subject matter were designed to reduce excessive cellular proliferation in vascular smooth muscle cells. Exemplary CycliCx peptides were designed for use in the vascular setting, in particular through application to the surface of stent materials as a novel therapeutic in balloon angioplasty and stent placement.

Peptides of the presently disclosed subject matter can also include modifications to enhance their transport across the membranes of SMCs. In some embodiments, the peptide comprises an N-terminal stearate modification. In some embodiments, the peptide is fused with another amino acid sequence that itself enhances transmembrane transport to create a fusion peptide. In some embodiments, the other amino acid sequence is an HIV-TAT sequence. An exemplary fusion peptide has the sequence YGRKKRRQRRRLDPAKDCGDQKYAY (SEQ ID NO: 16), wherein amino acids 1-11 represent an HIV-TAT peptide that serves a direct membrane penetration role and amino acids 12-25 represents an exemplary CycliCx peptide of the presently disclosed subject matter.

In some embodiments, a peptide of the presently disclosed subject matter is present as a component of a medical device. In some embodiments, the medical device is a stent that is coated with and/or otherwise capable of delivering a peptide of the presently disclosed subject matter to a desired target location.

The peptides described herein can be applied to stents that have been coated with a polymeric compound. Incorporation of one or more peptides of the presently disclosed subject matter into the polymeric coating of the stent can be carried out by dipping the polymer-coated stent into a solution containing the compound or drug for a sufficient period of time (such as, for example, five minutes) and then drying the coated stent (e.g., air drying) for a sufficient period of time (such as, for example, 30 minutes). The polymer-coated stent containing the peptide(s) of the presently disclosed subject matter can then be delivered to a vessel by any appropriate process, including but not limited to deployment from a balloon catheter. The production and loading of stents with active agents is described, for example, in U.S. Patent Application Publication Nos. 2020/00197371, 2020/0179574, 2020/0113717, 2020/0108232, 2020/0101049, 2020/0000975, 2020/0016299, 2019/0298802, 2019/0193109, and 2019/0117854, each of which is incorporated in its entirety.

In addition to stents, other devices that can be used to introduce the peptides of the presently disclosed subject matter to the vasculature include, but are not limited to grafts, catheters, and balloons.

The peptides described herein for use in polymer-coated stents can be used in combination with other pharmacological agents. The pharmacological agents that can be employed in combination with the peptides of the presently disclosed subject matter can in some embodiments be effective in preventing neointimal formation and/or supporting prevention of neointimal formation and sequelae thereof can be in some embodiments anti-proliferative agents, in some embodiments anti-platelet agents, in some embodiments anti-inflammatory agents, in some embodiments anti-thrombotic agents, and in some embodiments thrombolytic agents. By way of example and not limitation, anti-proliferative agents can be anti-mitotic agents that inhibit or affect cell division, such as but not limited to vinca alkaloids. Representative examples of vinca alkaloids include, but are not limited to, vincristine, paclitaxel, etoposide, nocodazole, indirubin, and anthracycline derivatives, such as, for example, daunorubicin, daunomycin, and plicamycin. Other anti-mitotic agents include anti-mitotic alkylating agents, such as, for example, tauromustine, bofumustine, and fotemustine, and anti-mitotic metabolites, such as, for example, methotrexate, fluorouracil, 5-bromodeoxyuridine, 6-azacytidine, and cytarabine.

Anti-platelet agents are therapeutic entities that act by inhibiting adhesion of platelets to surfaces, typically thrombogenic surfaces; and/or inhibiting aggregation of platelets; and/or inhibiting activation of platelets; and/or combinations thereof. Anti-platelet agents that can act as inhibitors of adhesion of platelets include, but are not limited to, eptifibatide, tirofiban, RGD (Arg-Gly-Asp)-based peptides that inhibit binding to gpIIbIIIa or avf33, antibodies that block binding to gpIIaIIIb or αvβ3, anti-P-selectin antibodies, anti-E-selectin antibodies, peptides that block P-selectin or E-selectin binding to their respective ligands, saratin, and anti-von Willebrand factor antibodies. Agents that inhibit ADP-mediated platelet aggregation include, but are not limited to, disagregin and cilostazol.

Anti-inflammatory agents can also be used in conjunction with the peptides of the presently disclosed subject matter, and can be deployed separately from the medical devices that comprise the peptides or can be added to said medical devices. Examples of these include, but are not limited to, prednisone, dexamethasone, hydrocortisone, estradiol, and non-steroidal anti-inflammatories, such as, for example, acetaminophen, ibuprofen, naproxen, and sulindac. Additional examples include those that inhibit binding of cytokines or chemokines to the cognate receptors to inhibit pro-inflammatory signals transduced by the cytokines or the chemokines. Representative examples of these agents include, but are not limited to, anti-IL1, anti-IL2, anti-IL3, anti-IL4, anti-IL8, anti-IL15, anti-GM-CSF, and anti-TNF antibodies.

The compositions and medical devices of the presently disclosed subject matter can also include anti-thrombotic agents such as, but not limited to, small molecules that inhibit the activity of factor Xa, heparinoid-type agents that can inhibit both FXa and thrombin, either directly or indirectly, such as, for example, heparin, heparan sulfate, low molecular weight heparins, and/or synthetic oligosaccharides. Anti-thrombotic agents also include direct thrombin inhibitors, such as, for example, melagatran, ximelagatran, argatroban, inogatran, and peptidomimetics of binding site of the Phe-Pro-Arg fibrinogen substrate for thrombin. Another class of anti-thrombotic agents that can be delivered are factor VII/VIIa inhibitors, such as, for example, anti-factor VII/VIIa antibodies, rNAPc2, and tissue factor pathway inhibitor (TFPI).

Thrombolytic agents, which may be defined as agents that help degrade thrombi (clots), can also be used as adjunctive agents, because the action of lysing a clot helps to disperse platelets trapped within the fibrin matrix of a thrombus. Representative examples of thrombolytic agents include, but are not limited to, urokinase or recombinant urokinase, pro-urokinase or recombinant pro-urokinase, tissue plasminogen activator or its recombinant form, and streptokinase.

Other drugs that can be used in combination with the peptides of the presently disclosed subject matter include cytotoxic drugs, such as but not limited to apoptosis inducers (e.g., TGF) and topoisomerase inhibitors such as but not limited to 10-hydroxycamptothecin, irinotecan, and doxorubicin. Other classes of drugs that can be used in combination with the peptides of the presently disclosed subject matter are drugs that inhibit cell de-differentiation and cytostatic drugs.

When used in the context of the presently disclosed subject matter, a medical device such as a stent can include a coating. In some embodiments, the coating can comprise, consist essentially of, or consist of any polymeric material in which the therapeutic agent, i.e., one or more peptides of the presently disclosed subject matter, is substantially soluble. In some embodiments, a purpose of the coating is to serve as a controlled release vehicle for the one or more peptides and/or other active agent(s) or as a reservoir for the one or more peptides and/or other active agent(s) to be delivered at the desired site where reduction or prevention of neointimal formation is desired. The coating can be polymeric and can further be hydrophilic, hydrophobic, biodegradable, or non-biodegradable. The material for the polymeric coating can be selected from the group consisting of polycarboxylic acids, cellulosic polymers, gelatin, polyvinylpyrrolidone, maleic anhydride polymers, polyamides, polyvinyl alcohols, polyethylene oxides, glycosaminoglycans, polysaccharides, polyesters, polyurethanes, silicones, polyorthoesters, polyanhydrides, polycarbonates, polypropylenes, polylactic acids, polyglycolic acids, polycaprolactones, polyhydroxybutyrate valerates, polyacrylamides, polyethers, and mixtures and copolymers of the foregoing. Coatings prepared from polymeric dispersions such as polyurethane dispersions, and acrylic acid latex dispersions can also be used with the therapeutic agents of the present invention.

Biodegradable polymers that can be used in this invention include polymers such as poly(L-lactic acid), poly(DL-lactic acid), polycaprolactone, poly(hydroxy butyrate), polyglycolide, poly(dioxanone), poly(hydroxy valerate), polyorthoester; copolymers such as poly(lactide-co-glycolide), polyhydroxy(butyrate-co-valerate), polyglycolide-co-trimethylene carbonate; polyanhydrides; polyphosphoesters; polyphosphoesterurethanes; polyamino acids; polycyanoacrylates; biomolecules such as fibrin, fibrinogen, cellulose, starch, collagen and hyaluronic acid; and mixtures of the foregoing. Biostable materials that are suitable for use in this invention include polymers such as polyurethanes, silicones, polyesters, polyolefins, polyamides, polycaprolactam, polyimides, polyvinyl chloride, polyvinyl methyl ether, polyvinyl alcohol, acrylic polymers and copolymers, polyacrylonitrile, polystyrene copolymers of vinyl monomers with olefins (such as styrene acrylonitrile copolymers, ethylene methyl methacrylate copolymers, ethylene vinyl acetate), polyethers, rayons, cellulosics (such as cellulose acetate, cellulose nitrate, cellulose propionate, etc.), parylene and derivatives thereof; and mixtures and copolymers of the foregoing.

Another polymer that can be used in the presently disclosed subject matter is poly(MPC_{w}:LAMₓ:HPMA_{y}:TSMA_{z}) where w, x, y, and z represent the molar ratios of monomers used in the feed for preparing the polymer and MPC represents the unit 2-methacryoyloxyethylphosphorylcholine, LMA represents the unit lauryl methacrylate, HPMA represents the unit 2-hydroxypropyl methacrylate, and TSMA represents the unit 3-trimethoxysilylpropyl methacrylate. The peptide-impregnated stent can be used to maintain patency of a vessel while preventing or reducing neointima formation. The delivery of an active agent of the presently disclosed subject matter (e.g., a peptide) thus reduces the rate of and/or eliminates neointima formation, including neointima formation in and around the stent.

The ability of the compositions of the presently disclosed subject matter to reduce or eliminate neointima formation can be demonstrated according to any method, including but not limited to the methods described in Bunchman & Brookshire, 1991; Yamagishi et al., 1993; and Shichiri et al., 1991.

When used in the above or other treatments, a therapeutically effective amount of one of the compositions of the presently disclosed subject matter can be employed in pure form or, where such forms exist, in pharmaceutically acceptable salt, ester, and/or prodrug forms. Alternatively, the composition may be administered as a pharmaceutical composition containing the peptide(s) of interest in combination with one or more pharmaceutically acceptable excipients. The phrase “therapeutically effective amount” of the compound of the presently disclosed subject matter refers to a sufficient amount of the compound to treat disorders, at a reasonable benefit/risk ratio applicable to any medical treatment. It will be understood, however, that the total daily usage of the compositions of the presently disclosed subject matter will be decided by the attending physician within the scope of sound medical judgement. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than required to achieve the desired therapeutic effect and to gradually increase the dosage until the desired effect is achieved.

The total daily dose of the compositions of the presently disclosed subject matter administered to a human or other animal can range in some embodiments from about 0.01 to about 10 mg/kg/day. For purposes of oral administration, doses can be in the range of in some embodiments from about 0.001 to about 3 mg/kg/day. For the purposes of local delivery from a stent, the daily dose that a patient will receive depends on the length of the stent. For example, a 15 mm stent can in some embodiments contain a peptide in an amount ranging from about 1 to about 120 micrograms and can deliver that peptide over a time period ranging from several hours to several weeks. If desired, the effective daily dose may be divided into multiple doses for purposes of administration; consequently, single dose compositions can contain such amounts or sub-multiples thereof to make up the daily dose. Topical administration can involve doses ranging from in some embodiments 0.001 to 3% mg/kg/day, depending on the site of application.

The pharmaceutical compositions of the presently disclosed subject matter comprise, consist essentially of, or consist of one or more peptides of the presently disclosed subject matter and a pharmaceutically acceptable carrier or excipient, which can be administered orally, rectally, parenterally, intracisternally, intravaginally, intraperitoneally, topically (as by powders, ointments, drops, gels, or transdermal patch), bucally, as an oral or nasal spray, or locally, which in some embodiments can be in a stent placed within the vasculature. The phrase “pharmaceutically acceptable carrier” means a non-toxic solid, semi-solid or liquid filler, diluent, encapsulating material or formulation auxiliary of any type. The term “parenteral,” as used herein, refers to modes of administration which include intravenous, intraarterial, intramuscular, intraperitoneal, intrasternal, subcutaneous and intraarticular injection, infusion, and placement, such as, for example, in vasculature.

Pharmaceutical compositions of the presently disclosed subject matter for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions can also contain adjuvants such as preservatives, wetting agents, emulsifying agents, and dispersing agents. Prevention of the action of microorganisms can be provided by the inclusion of various antibacterial and antifungal agents, for example, paraben, chlorobutanol, phenol sorbic acid, and the like. It can also be desirable to include isotonic agents such as sugars, sodium chloride, and the like. Prolonged absorption of the injectable pharmaceutical form can be brought about by the inclusion of agents that delay absorption such as aluminum monostearate and gelatin.

In some cases, in order to prolong the effect of an active agent (e.g., a peptide), it can be desirable to slow the absorption of the peptide from subcutaneous or intramuscular injection. This can be accomplished by the use of a liquid suspension of crystalline or amorphous material with poor water solubility. The rate of absorption of the agent then depends upon its rate of dissolution which, in turn, can depend upon size and form. Alternatively, delayed absorption of a parenterally administered peptide can be accomplished by dissolving or suspending the peptide in an oil vehicle.

Injectable depot forms are made by forming microencapsule matrices of one or more peptides of the presently disclosed subject matter in biodegradable polymers such as polylactide-polyglycolide. Depending upon the ratio of peptide to polymer and the nature of the particular polymer employed, the rate of peptide release can be controlled. Examples of other biodegradable polymers include poly(orthoesters) and poly(anhydrides). Depot injectable formulations are also prepared by entrapping the peptide(s) in liposomes or microemulsions which are compatible with body tissues.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

The active agents of the presently disclosed subject matter (e.g., peptides) can also be in micro-encapsulated form, if appropriate, with one or more of the above-mentioned excipients.

Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agaragar, and tragacanth, and mixtures thereof.

Topical administration includes administration to the skin or mucosa, including surfaces of the lung and eye. Compositions for topical administration, including those for inhalation, may be prepared as a dry powder which may be pressurized or non-pressurized. In non-pressurized powder compositions, the active ingredient in finely divided form may be used in admixture with a larger-sized pharmaceutically acceptable inert carrier comprising particles having a size, for example, of up to 100 micrometers in diameter. Suitable inert carriers include sugars such as lactose. In some embodiments, at least 95% by weight of the particles of the active ingredient have an effective particle size in the range of 0.01 to 10 micrometers. Compositions for topical use on the skin can in some embodiments also include ointments, creams, lotions, and gels.

Alternatively, the composition may be pressurized and contain a compressed gas, such as nitrogen or a liquified gas propellant. The liquified propellant medium and indeed the total composition is in some embodiments such that the active ingredient does not dissolve therein to any substantial extent. The pressurized composition may also contain a surface active agent. The surface active agent may be a liquid or solid non-ionic surface active agent or may be a solid anionic surface active agent. It is preferred to use the solid anionic surface active agent in the form of a sodium salt.

Compounds of the presently disclosed subject matter can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a peptide of the presently disclosed subject matter, stabilizers, preservatives, excipients, and the like.

Exemplary lipids are the phospholipids and the phosphatidyl cholines (lecithins), both natural and synthetic. Methods to form liposomes are known in the art. See e.g., Prescott (ed.) (1976) Methods in Cell Biology, Volume XIV, Academic Press, New York, N.Y., United States of America, page 33 et seq.

### III. Representative Methods and Uses

The compositions of the presently disclosed subject matter can be employed for various purposes related to inhibiting and/or preventing undesirable smooth muscle cell (SMC) proliferation. As used herein, the phrase “undesirable smooth muscle cell proliferation” relates to any proliferation of smooth muscle cells which result in an undesirable outcome in vivo, ex vivo, and/or in vitro.

In some embodiments, the undesirable SMC proliferation results in neointima formation. As such, in some embodiments the presently disclosed subject matter relates to methods for inhibiting neointima formation in mammals. In some embodiments, the methods comprise, consist essentially of, or consist of contacting a vascular smooth muscle cell (SMC) or a plurality of smooth muscle cells (SMCs) with an effective amount of a composition comprising, consisting essentially of, or consisting of a peptide or a combination of peptides as disclosed herein. In some embodiments, the SMC is a vascular SMC. In some embodiments, the vascular SMC is present in a subject. In some embodiments, the vascular SMC is present in a blood vessel in a subject.

As described herein above, the composition can comprise, consist essentially of, or consist of a peptide or a combination of peptides as disclosed herein, or the composition can comprise, consist essentially of, or consist of a peptide or a combination of peptides as disclosed herein complexed with a carrier such as but not limited to a medical device.

In some embodiments, the composition comprises, consists essentially of, or consists of an effective amount of a peptide or combination of peptides as described herein present within and/or coated onto a stent, which in some embodiments can be formulated to release the peptide or combination of peptides from the stent when in contact with a vascular SMC. In some embodiments, the peptide or combination of peptides are formulated to release from the stent over time, which in some embodiments can be selected from the group consisting of one or more minutes, one or more hours, one or more days, one or more weeks, and one or more months.

More generally, the presently disclosed subject matter relates in some embodiments to methods for inhibiting division and/or proliferation of vascular SMCs. In some embodiments, the methods comprising, consist essentially of, or consist of contacting one or more vascular SMCs with an effective amount of a peptide or combination of peptides as disclosed herein. In some embodiments, the vascular SMCs are present in a subject, which is optionally a mammal, and further optionally a human.

In some embodiments, proliferation of the vascular SMCs is associated with a cardiovascular disease, disorder, or condition in a subject. In some embodiments, proliferation of the vascular SMCs results in neointima formation, and the compositions and methods of the presently disclosed subject matter inhibit, reduce, or prevent the neointima formation. As such, in some embodiments the presently disclosed subject matter relates to uses of compositions that comprise, consist essentially of, or consist of one or more of the peptides disclosed herein for treating cardiovascular diseases, disorders, and/or conditions associated with undesirable vascular SMC proliferation, wherein the undesirable vascular SMC proliferation is optionally characterized by and/or results at least in part from neointima formation.

Alternatively or in addition, in some embodiments the presently disclosed subject matter relates to uses of compositions that comprise, consist essentially of, or consist of one or more of the peptides disclosed herein for inhibiting neointima formation in mammals, optionally humans.

In some embodiments, proliferation of the vascular SMCs is associated with a cancer and/or tumor growth and/or progression in a subject. As would be understood by one of ordinary skill in the art, growth and/or progression of cancers and tumors can be associated with undesirable SMC proliferation, and thus the compositions and methods of the presently disclosed subject matter can be used to reduce, inhibit, or prevent the growth and/or progression of cancers and/or tumors. In some embodiments, the cancer is selected from the group consisting of breast cancer, in some embodiments triple negative breast cancer, and in some embodiments the cancer is melanoma. In some embodiments, the cancer is basal cell carcinoma. In some embodiments, a composition that is intended for use in melanoma or another skin cancer (such as but not limited to basal cell carcinoma) can be formulated as a cream, gel, or ointment for topical administration.

The peptides of the presently disclosed subject matter can also be employed as part of a process for high throughput small molecule screening of cyclin E/Cx43 disruptors. In some embodiments, a cell-free system is employed to produce active cyclin E polypeptides and for use with synthesized CycliCx peptides, for which previously validated protocols are disclosed in Johnstone et al., 2012. These allow for high throughput arrays to define effective small molecules without the complication of cell-based assays.

For example, in some embodiments the presently disclosed subject matter relates to methods for identifying inhibitors of a cyclin E/connexin43 interaction comprising (a) combining a cyclin E polypeptide and a connexin43 polypeptide in the presence of one or more peptides as described herein under conditions sufficient to induce formation of a complex between the cyclin E polypeptide and the connexin43 polypeptide; and (b) determining an extent of binding of the one or more peptides to the complex in the presence and the absence of a candidate inhibitor of a cyclin E/connexin43 interaction. In some embodiments, a reduction in binding of the one or more peptides to the complex in the presence of the candidate inhibitor of a cyclin E/connexin43 interaction as compared to in the absence of the candidate inhibitor of a cyclin E/connexin43 interaction is indicative of the candidate inhibitor of a cyclin E/connexin43 interaction being an inhibitor of a cyclin E/connexin43 interaction. Any method that can be employed to assay competitive interactions between molecules can be employed in the screening methods of the presently disclosed subject matter. In some embodiments, determining step comprises employing a Förster resonance energy transfer (FRET) based method (see e.g., U.S. Pat. No. 5,342,789, incorporated by reference herein in its entirety, and Cardullo et al., 1988). By way of example and not limitation, a peptide of the presently disclosed subject matter can be fluorescently labeled and a reduction in FRET signal can be assayed to assess relative binding of the peptide of the presently disclosed subject matter to a cyclin E polypeptide in the presence of a candidate inhibitor.

Any molecule that could potentially be an inhibitor of a cyclin E/connexin43 interaction can be tested using the compositions and methods of the presently disclosed subject matter. In some embodiments, a candidate inhibitor is a small molecule. In some embodiments, a candidate inhibitor is an antibody or an antigen-binding fragment thereof.

As such, in some embodiments the presently disclosed subject matter also provides uses of a peptide as described herein for identifying an inhibitor of a cyclin E/connexin43 interaction, wherein the peptide of claim 1 or claim 2 is employed in a competition assay with one or more candidate inhibitors of a cyclin E/connexin43 interaction.

### EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Materials and Methods for the EXAMPLES

Peptide array. The C-terminus of the human Cx43 polypeptpide (Cx43-CT) is about 150 amino acids (a.a.) with a high abundance (>10%) of post-translationally modified sites (Aasen et al., 2018). This means that hundreds of combinations are required to delineate binding parameters. Using novel peptide array technology, the sites of interaction between connexin 43 (Cx43) and cyclin E were mapped. Immobilized peptide libraries corresponding to 25-residue regions of Cx43-CT were designed, synthesized, and nitrocellulose arrays generated. To test the requirement for phosphorylation, specific residues were replaced to mimic known protein interacting states (e.g., MAPK-Cx43 serines 255/262/279/282; see Johnstone et al. 2012). The role for phosphorylation was assayed in binding using single and multiple residue replacements. These included wild-type serine (S), native phospho-serine (pS), aspartate (D, phospho-mimic), or alanine (A, phospho-null) residues. Recombinant human cyclin E protein was generated for incubation with arrays (Johnstone et al. 2012). Arrays were bathed in 100% ethanol, equilibrated in Trist Buffered Saline plus TWEEN® 20 (TBST) and blocked in TBST containing 5% milk for 2 hours at room temp. The cyclin E protein, in TBST-5% milk, was incubated with membranes at 10 μg/mL in the cold room overnight. Membranes were washed in TBST, incubated with anti-cyclin E primary antibody, and developed with anti-rabbit HRP secondary antibody (Sigma Aldrich, St. Louis, Mo., United States of America). Signal was then detected using electrochemiluminescence (ECL) detection reagents as per manufacturer protocol (PIERCE™ brand, available from Thermo Fisher Scientific, Inc., Waltham, Mass., United States of America) and blots were scanned for analysis. To define critical residues and minimal binding regions within the identified binding sites, array-based site directed alanine mutagenesis and truncation mutation arrays following the above protocol for cyclin E binding were performed.

Protein expression and interaction in cultured human coronary artery smooth muscle cells. Human coronary artery smooth muscle cells (Thermo Fisher Scientific, Inc., Waltham, Massachusetts, United States of America), were cultured until confluent in M231 media containing growth supplement (Thermo Fisher Scientific, Low Serum Growth Supplement Kit (LSGS) kit) as well as 20% fetal bovine serum (FBS). Prior to incubations, media was switched to low serum media (M231+2% FBS) for 3 days as previously described (Lohman et al., 2016). Cells were then incubated with oxidized phospholipids 1-palmitoyl-2-glutaroyl-sn-glycero-3-phosphorylcholine (PGPC, 100 μg) and 1-palmitoyl-2-oxovaleroyl-sn-glycero-3-phosphorylcholine (POVPC 100 μg), or the growth factor platelet derived growth factor beta (PDGF, 20 ng) as we have previously described (Johnstone et al. 2009; Johnstone et al. 2012) for 48 hours prior to harvesting and analysis for Western blotting and co-immunoprecipitation and EDU proliferation by flow cytometry as previously described (Johnstone et al. 2012). For peptide incubations, peptides were pre-incubated for 30 minutes with cells in fresh media prior to adding any further treatments.

Mouse carotid surgeries. Carotid ligation and pluronic application surgeries were performed as described (Johnstone et al. 2009; Johnstone et al. 2012). Briefly, mice were anesthetized by isoflurane and the left common carotid artery was exposed and carefully freed from surrounding tissue. A 6/0 silk suture was run under the carotid artery distal to the bifurcation site and two consecutive, flat square knots were tied tight enough to restrict blood flow. Control mice were surgical controls, where carotid artery was exposed and freed of surrounding tissue as described above but no suture was applied. Following ligation either 35% F127 pluronic gel, CycliCx peptide (200 μg in pluronic; LDPAKDCGDQKYAY; SEQ ID NO: 1) or control peptide (200 μg in pluronic; AYFNGCSSPTAPLDP; SEQ ID NO: 3) was applied to the outside of the carotid wall and surgical wound closed. At 2 weeks after surgery, mice were euthanized with an intraperitoneal injection of 60-90 mg/kg pentobarbital. Mice were perfused with 4% paraformaldehyde (PFA) and both treated (left common) and control (right common) carotids removed and stored in 4% PFA overnight for whole mount analysis or immunosectioning. For in vivo cell proliferation studies, mice were preinjected with 5-ethynyl-2′-deoxyuridine (EDU) prior to surgery and carotids were removed post-treatment for whole mount or tissue cross section analysis of EDU incorporation into proliferating cells. For analysis of neointima formation in vessels, areas corresponding to the media (containing vascular smooth muscle cells (VSMC)), neointima (containing proliferating VSMC) and lumen (void area) were measured on Haemotoxylin and Eosin (H&E) stained cross sections of carotid vessels and analyzed by Image J, image analysis software. The ratio of media:neointima was calculated.

Ex vivo, human saphenous vein explant model (HUSVEM). The translational relevance of the peptides was assessed in an ex vivo, human saphenous vein explant model (HUSVEM) based on previously validated protocols (Baker et al. 1997; Dreifaldt et al. 2013; Loesch, Dashwood & Souza, 2006; George et al., 1996). Briefly, human veins were obtained as excess tissue from coronary artery bypass surgeries. The protocol was assessed by the Institutional Review Board (IRB) of the University of Virginia (Charlottesville, Va., United States of America), and no IRB protocol was required. Human saphenous veins were opened laterally, cut into 10 mm sections, and pinned with the endothelial side facing up on SYLGARD® brand silicone curing agent-coated plates in RMPI media containing 2% FBS. After 1 hour, cells were incubated with either control peptide or CycliCx peptide (30 μM; LDPAKDCGDQKYAY; SEQ ID NO: 1) or everolimus (10 nM; Sigma Aldrich) for a further 60 minutes, prior to adding PDGF (50 ng/mL). Media was changed daily with fresh media containing peptides or everolimus and PDGF. At the end of 14 days media was removed and tissues fixed in 4% PFA for 24 hours then washed in 70% ethanol, prior to embedding in 3% agarose. Samples were then processed by routine paraffin embedding and stained using hematoxylin and eosin and van Gieson stains. Comparisons of neointmal growth were made, where the uppermost layer of elastin was used to identify the start region for formed neointima. Neointimal areas were calculated across 10 serial sections from each tissue and averaged. Analysis of PDGF treated vessels to peptide and everolimus treated tissues were made.

### Example 1

### Peptide Array Experiments

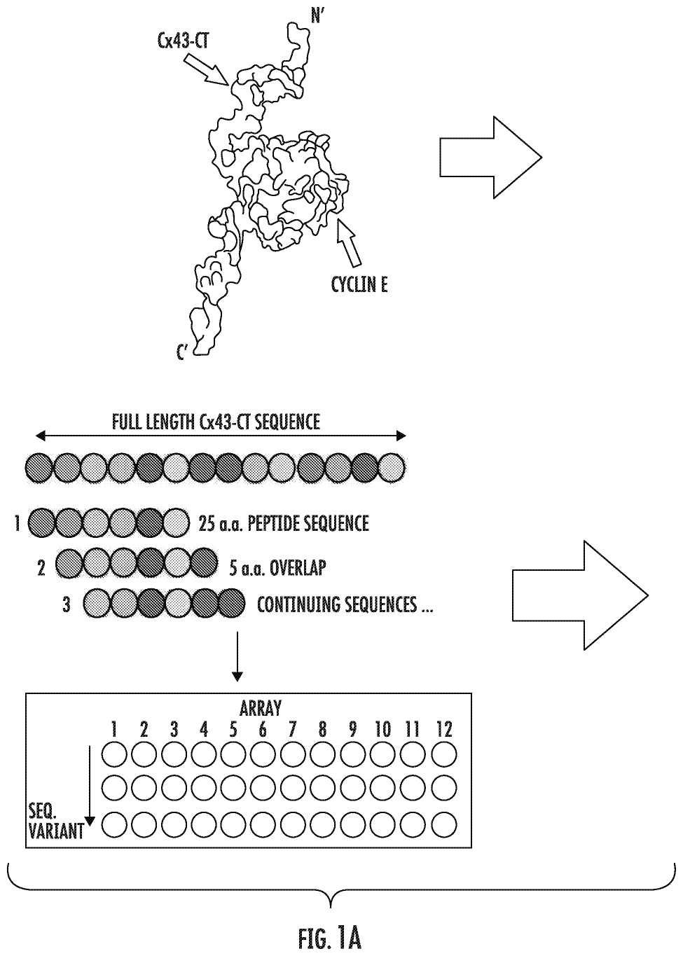

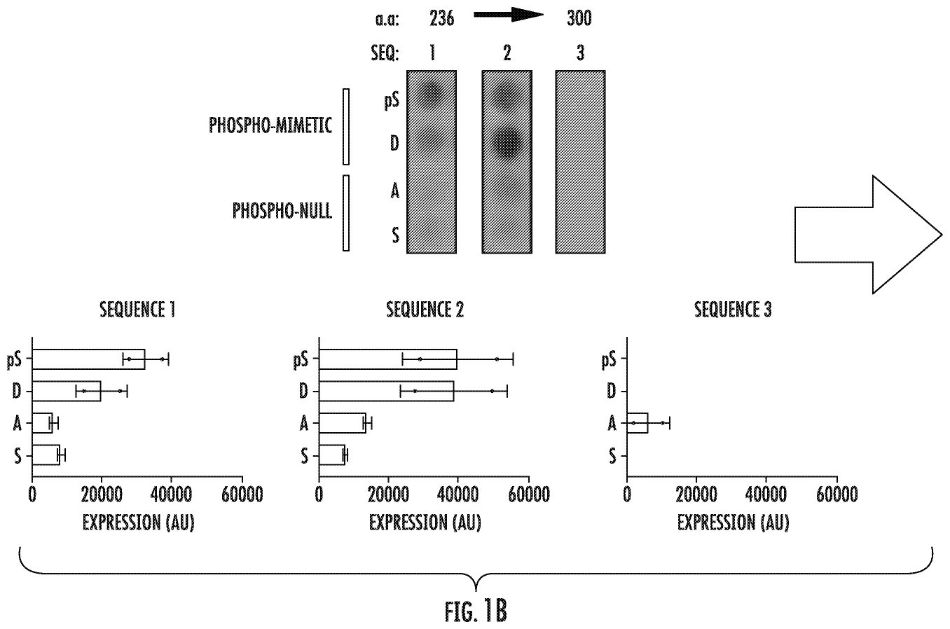

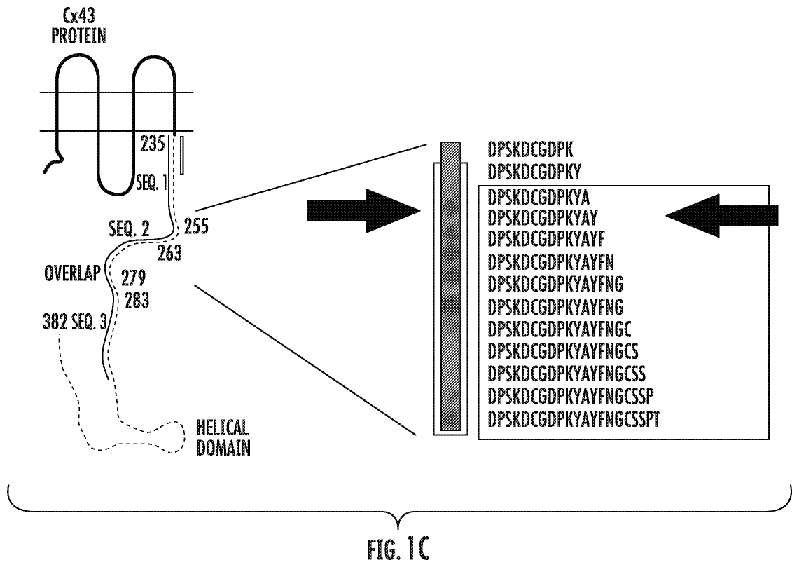

Peptide array experiments were performed as described herein above, and the results are presented in FIGS. 1A-1C.

In FIG. 1A, a potential interaction schematic for the full length cyclin E and the Cx43-C-terminus is depicted. The predicted interaction model was created in silico using a structural model of Cx43-CT and cyclin E (PDB ID: Cx43-1R5S & CCNE-1W98), processed with FireDock (Mashiach et al., 2008) and PatchDock (Schneidman-Duhovny et al., 2005). FIG. 1A presents a schematic of an array, which highlights the arrangement for printing peptide sequences on nitrocellulose, with overlapping sequences (corresponding to the full length Cx43 C-terminal mouse sequence (Accession No. NP 034418.1 from residues 234 to 382)). Resulting array blots from these experiments (FIG. 1B), demonstrated that phosphorylation (ps/D) at critical residues was required for cyclin E binding with the array. Multiple sequences were identified (Sequence 1-3; which were arbitrarily labelled based on identification of 25 amino acid sequences to be tested for truncation and mutagenesis studies) on Cx43 C-terminus. In FIG. 1C, a truncation-based array demonstrated the minimum sequence (based on mouse Cx43 sequence; indicated with a left-facing arrow; DPSKDCGDPKYAY (SEQ ID NO: 7) sequence that was required for binding. This sequence formed the basis of the CyClix peptide, which was similar, with the exception of a serine to alanine switch at residue 3 and proline to glutamine at residue 9. The sequence was further extended at the N-terminus with addition of a leucine (L) residue based on the chemistry for attaching stearate groups to the peptide sequence to generate the exemplary CyClix peptide of SEQ ID NO: 1.

Additional peptides containing either the mouse CyClix peptide (LDPSKDCGDPKYAY; SEQ ID NO: 2) or the human CyClix peptide (LDPAKDCGDQKYAY; SEQ ID NO: 1) with alterations in phosphorylated regions (e.g., aspartate instead of serine) are also tested. All peptides generated contained an N-terminal stearyl group and were synthesized by a peptide service (Thermo Fisher Scientific).

### Example 2

### Protein Interaction Studies in Human Coronary Artery Smooth Muscle Cells (SMCs)

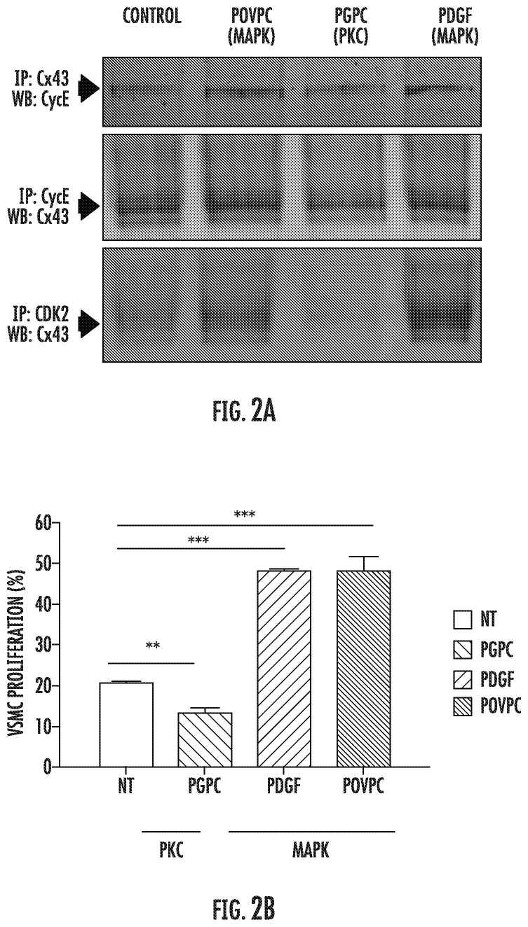

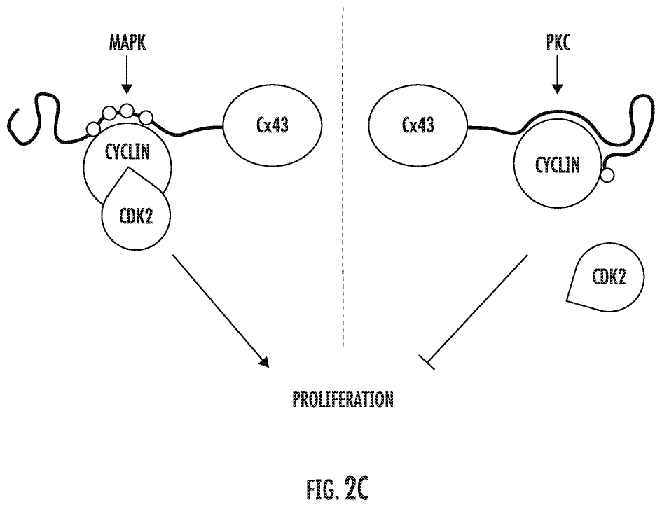

Human smooth muscle cells in culture were treated with agents that have been found to promote MAPK phosphorylation of Cx43 (POVPC and PDGF) or with an agent that causes PKC phosphorylation of Cx43 (PGPC). These phosphorylation changes are reported to be exclusive and do not occur at the same time. Co-immunoprecipitation data showed that binding with cyclin E occurred under these conditions. However, the ability to bind with, and therefore activate, cyclin D was inhibited in the presence of PGPC (see FIG. 2A). These alterations in phosphorylation corresponded with increased proliferation for PDGF/POVPC and decreased proliferation for PGPC as demonstrated by flow cytometry of EDU incorporation (see FIG. 2B). Taken together, this identified Cx43 phosphorylation status as a molecular switch that controlled cellular proliferation (MAPK-Cx43) or quiescence (PKC-Cx43), a model that is depicted in see FIG. 2C.

### Example 3

### Peptide Treatment of Human Coronary Artery SMCs

Peptides representing sequences within the C-terminus of human Cx43, designed by peptide array, inhibited proliferation of human smooth muscle cells (see FIGS. 3A-3F). Cells were pretreated for 30 minutes with peptides, prior to incubation with PDGF for 2 days. This resulted in significantly reduced EDU incorporation (proliferation) by comparison to PDGF alone or control (Scr) peptide (FIGS. 3A and 3B). Treatment with an exemplary CycliCx peptide (pep3(mouse, LDPSKDCGDPKYAY; SEQ ID NO: 2) significantly altered the phosphorylation status of Cx43 as shown by western blot (see FIGS. 3C and 3D). In particular, CycliCx predominantly induced the PKC-phosphorylated Cx43 species, which has been shown to be linked with reduced proliferation (see FIGS. 3C and 3D). Co-immunoprecipitation studies in human SMC demonstrated that the CycliCx peptide (CxPep) significantly reduced interactions with cyclin E (see FIGS. 3E and 3F; LDPSKDCGDPKYAY: SEQ ID NO: 2).

### Example 4

### Peptide Treatment of Mouse Carotid Arteries

An exemplary CycliCx peptide (LDPAKDCGDQKYAY; SEQ ID NO: 1) was applied to the carotids of mice for 14 days after placement of a suture to induce neointimal formation (FIG. 4A). In both ligation only and ligation with peptide, extensive neointima formed. This was completely inhibited in mice using the CycliCx peptide (see FIG. 4B and Table 1; LDPAKDCGDQKYAY; SEQ ID NO: 1). Effects were tested in both male and female mice, and no sex-specific differences were noted (FIG. 4B and Table 1).

TABLE 1
Statistical Analysis of Neointimal Formation

| | | N | Neointimal/media | p Value to Pluronic |
|---|---|---|---|---|
| Sex | Treatment | value | Ratio (±SEM) | No Ligation |
| Male | Pluronic No | 12 | 0.0000 ± 0.0000 | n.a. |
| | Ligation | | | |
| | Pluronic + | 8 | 0.5425 ± 0.0892 | <0.0001 |
| | Ligation | | | |
| | Control Pep + | 10 | 0.6690 ± 0.1111 | <0.0001 |
| | Ligation | | | |
| | CycliCx + | 10 | 0.0110 ± 0.0055 | <0.9993 |
| | Ligation | | | |
| Female | Pluronic + | 3 | 0.0000 ± 0.0000 | n.a. |
| | Ligation | | | |
| | Control Pep + | 3 | 1.017 ± 0.3869 | 0.0326 |
| | Ligation | | | |
| | CycliCx + | 3 | 0.0060 ± 0.0379 | 0.9742 |
| | Ligation | | | |

n.a.: not applicable;
ns: not significant

### Example 5

### Peptide Treatment of Human Saphenous Vein Tissues

An exemplary CycliCx peptide (LDPAKDCGDQKYAY; SEQ ID NO: 1) was applied to human saphenous veins in ex vivo preparations for 2 weeks in the presence of PDGF and compared to everolimus, an agent used in drug eluting stents. In PDGF and control peptide, neointima were identified after 14 days, whereas both everolimus and CycliCx inhibited neointimal formation (see FIGS. 5A and 5B).

### Example 6

### MTT Assay of Cancer Cell Proliferation

Two cancer cell lines originating from human melanoma (A375-MA2) and triple negative breast cancer (Hs578T) were used to assess the role of CycliCx in inhibiting proliferation. 500 cells per well were cultured in a 96-well plate containing 100 μl of medium. The next day (Day 0), 30 μM of CycliCx peptide or control peptide was added to the cells. Starting from Day 0, 10 μl of WST-1 reagent (viability/MTT) was added to each well and incubated for 75 minutes. Absorbance was measured at 450 nm. Data are presented in FIGS. 6B (A375-MA2) and 6C (Hs578T) as mean±SEM. p<0.05 by two way ANOVA.

As shown in FIG. 6A, both human melanoma and breast cancer cells abundantly expressed Cx43 in multiple phosphorylation states. The expression of Cx43 has previously been associated with proliferation in these cells. A single treatment of the cells with CycliCx peptide (30 μM) at day 0 was sufficient to significantly inhibit proliferation in both cell types.

### Example 7

### Small Molecule Screening of Cyclin E/Cx43 Disruptors

To test small molecule blockade of cyclin E/CycliCx protein interactions in a high throughput array, an AlphaScreen (Perkin Elmer) modified FRET based approach is employed. This assay has been widely used in the testing and identification of small molecule inhibitors (see e.g., Schorpp et al., 2014) and is readily adaptable to use with synthesized cyclin E protein and exemplary CycliCx peptides. Protein interactions are titrated to define the lowest nanomolar range for interaction, with binding detected on an Omega fluorescence plate reader. At these concentrations, proteins are combined in a 384 well plate format with panels of small molecule inhibitors. The Sigma LoPac small molecule panel contains 1280 pharmacologically active small molecules, marketed drugs, and pharmaceutically relevant structures that includes known cell cycle and kinase inhibitors. The assays are performed by sequentially adding small molecules, cyclin E protein (to permit binding site blockade), then CycliCx peptide to 384 well plates and using AlphaScreen donor/acceptor pair signals for cyclin E and CycliCx detected. Plating of proteins and small molecules 0.1-100 μM in triplicate is performed using the Mosquito HTS robot. All small molecules that reduce interactions (AlphaScreen signal) below a threshold of 50% of control values are processed for secondary screening using the TRUHIT kit (Perkin Elmer) designed to eliminate false positives.

It can be important to screen molecules for promiscuous effects with various filters e.g. Pan Assay Interfering Compounds (PAINS). However, it is not necessary, and could be potentially misleading (Baell & Nissink, 2018), to triage molecules at this stage. These analyses are performed at prior to treatment in mice. If molecules are suspected of being false positives, further screening using time resolved FRET analysis is employed to eliminate any “frequent hitter” that may occur (Schorpp et al., 2014).

A number of small molecules that disrupt the cyclin E/Cx43 may not be suitable based on cell permeability or cytotoxicity issues that reduce their smooth muscle cell specificity and overall utility.

To test their effects, identified small molecules are added to cultured human smooth muscle or endothelial cells plated in 384 well plates. Cells are stalled in cell cycle to reduce proliferation, then treated with PDGF to induce proliferation in the presence of the small molecules. Dose optimization is performed for each molecule with ranges between 0.1-100 11M tested. Cytotoxicity is assessed using a fluorescence-based active caspase 3 assay (Abcam). Cellular proliferation is assessed by of incorporation of 5-ethynyl-2′-deoxyuridine (EDU) to the nuclei of proliferating cells. The EDU signal is calibrated to the number of cells (DAPI, nuclei of all cells; see FIG. 7) and overall percentages calculated. Cyclin E/Cx43 interactions are detected in plated cells using the DuoLink fluorescence-based proximity ligation assay (Sigma). DuoLink can measure protein interactions up to 40 nm apart. Any loss of fluorescent signal indicates that small molecules are disrupting the PDGF-induced cyclin E/Cx43 signal (see FIG. 7). Both EDU and DuoLink assays are well suited to high throughput analysis in 384 well plates, and protocols for their detection have been developed (Johnstone et al., 2012).

All plates are scanned on the Operetta confocal scanner (shown in FIG. 7). The Operetta system, combined with a robotic arm, allows for high throughput screening of multi-well formats. The Operetta software is used for automatic detection of nuclei/EDU (proliferation) and for cell/DuoLink signal (protein interactions). Some or all of these parameters—toxicity (endothelial and smooth muscle); proliferation; and protein interactions—are combined to select small molecules for application in mouse and human studies.

Candidate small molecule cyclin E/Cx43 disruptors are tested in smooth muscle cell proliferation assays and mouse arteries and human saphenous vein tissue assays as described herein above.

### Discussion of the EXAMPLES

As disclosed herein, two proteins, connexin 43 (Cx43) and cyclin E, bind together in SMC in response to pathological stimuli. Once bound, these proteins promote SMC proliferation and neointimal formation in mice (Johnstone et al., 2012). It has also been shown that, by blocking the processes underlying these protein interactions, it is possible to inhibit neointimal formation. The CycliCx peptides disclosed herein compete with and block to Cx43 from interacting with cyclin E, thus reducing cellular proliferation.

Since initially characterizing a general interaction of the Cx43 and cyclin E proteins and their effects in disease (Johnstone et al., 2009; Johnstone et al., 2012), specific peptide arrays were employed to identify the short amino acid sequences where Cx43 and cyclin E proteins bind. Using these data, peptides representing modified sequences of Cx43 that were synthesized commercially were tested. In testing, exemplary peptide mimetics were identified and are described herein. One particular such peptide mimetic, referred to herein as CycliCx, showed specific effects in reducing vascular SMC proliferation.

Two models were developed for in vivo and ex vivo testing. The first is a carotid artery ligation neointimal model in mice, where the CycliCx peptide was applied to the carotid for 14 days. The second model used human saphenous veins in culture, to produce neointima, with CycliCx applied to surrounding media for 14 days. Using these models, it has been demonstrated that Cx43 interacts with cyclin E in human vascular tissues, and is associated with SMC proliferation and neointimal formation. In testing, the CycliCx peptide reduced human SMC proliferation and completely inhibited neointimal formation in mouse carotids and human saphenous veins.

### REFERENCES

All references listed in the instant disclosure, including but not limited to all patents, United States and PCT International patent applications and publications thereof, scientific journal articles, and database entries (including but not limited to Uniprot, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.

Aasen et al. (2017) Gap junctions and cancer: communicating for 50 years. Nat Rev Cancer. 17:74.

Baell & Nissink (2018) Seven Year Itch: Pan-Assay Interference Compounds (PAINS) in 2017-Utility and Limitations. ACS Chem Biol. 13:36-44.

Benedetto et al. (2016) State of the art in coronary revascularization: Everolimus eluting stents versus multiple arterial grafting. Int J Cardiol. 219:345-9.

Benedetto et al. (2018) New-generation stents compared with coronary bypass surgery for unprotected left main disease: A word of caution. J Thorac Cardiovasc Surg. 155(5):2013-2019.

Benjamin et al. (2017) Heart Disease and Stroke Statistics-2017 Update: A Report From the American Heart Association. Circulation. 135:e146-e603.

Bonaa et al. (2016) Drug-Eluting or Bare-Metal Stents for Coronary Artery Disease. N Engl J Med. 375:1242-52.

Bunchman & Brookshire (1991) Transplantation Proceed. 23:967-968.

Cardullo et al. (1988) Detection of nucleic acid hybridization by nonradiative fluorescence resonance energy transfer. Proc Natl Acad Sci USA. 85(23):8790-4.

Chadjichristos et al. (2006) Reduced connexin43 expression limits neointima formation after balloon distension injury in hypercholesterolemic mice. Circulation. 113:2835-43.

Escaned et al. (2016) Rationale and design of the SYNTAX II trial evaluating the short to long-term outcomes of state-of-the-art percutaneous coronary revascularisation in patients with de novo three-vessel disease. EuroIntervention. 12:e224-34.

GENBANK® Accession Nos. NP 000156.1 and NP 034418.1.

Hung et al. (2012) Remodeling of rabbit abdominal aorta and Cx43 gap junctions after stent placement: effect of balloon injury plus cholesterolenriched diet. Int Angiol. 31:62-9.

Johnstone et al. (2009) Oxidized phospholipid species promote in vivo differential cx43 phosphorylation and vascular smooth muscle cell proliferation. Am J Pathol. 175:916-24.

Johnstone et al. (2012) MAPK Phosphorylation of Connexin 43 Promotes Binding of Cyclin E and Smooth Muscle Cell Proliferation. Circulation Research. 111:201-U205.

Liao et al. (2007) Smooth muscle-targeted knockout of connexin43 enhances neointimal formation in response to vascular injury. Arterioscler Thromb Vasc Biol. 27:1037-42.

Loewenstein & Kanno (1966) Intercellular communication and the control of tissue growth: lack of communication between cancer cells. Nature. 209:1248-9.

Mashiach et al. (2008) FireDock: a web server for fast interaction refinement in molecular docking. Nucleic Acids Res. 36(Web Server issue):W229-W232.

Parasca et al. (2016) Incidence, Characteristics, Predictors, and Outcomes of Repeat Revascularization After Percutaneous Coronary Intervention and Coronary Artery Bypass Grafting: The SYNTAX Trial at 5 Years. JACC Cardiovasc Interv. 9:2493-2507.

Schneidman-Duhovny et al. (2005) PatchDock and SymmDock: servers for rigid and symmetric docking. Nucleic Acids Res. 33(Web Server issue):W363-W367.

Schorpp et al. (2014) Identification of Small-Molecule Frequent Hitters from AlphaScreen High-Throughput Screens. J Biomol Screen. 19:715-26.

Shichiri et al. (1991) J. Clin. Invest. 87:1867-1871

Sun et al. (2017) Effects of elemene on inhibiting proliferation of vascular smooth muscle cells and promoting reendothelialization at the stent implantation site. Biomater Sci. 5:1144-1155.

U.S. Patent Application Publication Nos. 2003/0017534, 2018/0298087, 2018/0312588, 2018/0346564, 2019/0117854, 2019/0151448, 2019/0193109, 2019/0298802, 2020/0000975, 2020/0016299, 2020/00197371, 2020/0101049, 2020/0108232, 2020/0113717, 2020/0179574.

U.S. Pat. Nos. 4,704,692; 4,816,567; 4,946,778; 5,342,789; 5,482,856; 6,479,284; 6,677,436; 7,060,808; 7,906,625; 8,398,980; 8,436,150; 8,796,439; 10,253,111.

Yamagishi et al. (1993) Biochem. Biophys. Res. Comm. 191:840-846.

Zhang et al. (2016) Phenotypic transformation of smooth muscle cells from porcine coronary arteries is associated with connexin 43. Mol Med Rep. 14:41-8.

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

1. A peptide comprising, consisting essentially of, or consisting of the amino acid sequence LDPAKDCGDQKYAY (SEQ ID NO: 1) or LDPSKDCGDPKYAY (SEQ ID NO: 2).

2. The peptide of claim 1, wherein the peptide comprises an N-terminal stearate modification.

3. A method for inhibiting neointima formation in a mammal, the method comprising contacting a vascular smooth muscle cell (SMC) with an effective amount of a composition comprising, consisting essentially of, or consisting of the peptide of claim 1, the peptide or claim 2, or a combination thereof.

4. The method of claim 3, wherein the vascular SMC is present in a subject.

5. The method of claim 3, wherein the vascular SMC is present in a blood vessel in a subject.

6. The method of claim 3, wherein the effective amount of the peptide of claim 1 is present within and/or coated onto a stent and is formulated to release from the stent when in contact with a vascular SMC.

7. The method of claim 6, wherein the peptide of claim 1 is formulated to release from the stent over a course of time selected from the group consisting of one or more minutes, one or more hours, one or more days, one or more weeks, and one or more months.

8. A method for inhibiting division and/or proliferation of a vascular SMC, the method comprising contacting the vascular SMC with an effective amount of the peptide of claim 1.

9. The method of claim 8, wherein the vascular SMC is present in a subject.

10. The method of claim 8, wherein the vascular SMC is present in a blood vessel in a subject.

11. The method of claim 8, wherein the effective amount of the peptide of claim 1 or claim 2 is present within and/or coated onto a stent.

12. The method of claim 22, wherein the peptide of claim 1 is formulated to release from the stent over a course of time selected from the group consisting of one or more minutes, one or more hours, one or more days, one or more weeks, and one or more months.

13. Use of the peptide of claim 1 for treating a cardiovascular disease or disorder associated with undesirable vascular SMC proliferation, optionally wherein the undesirable vascular SMC proliferation is characterized by and/or results at least in part from neointima formation.

14. Use of the peptide of claim 1 for inhibiting neointima formation in a mammal.

15. A peptide for use in treating a cardiovascular disease or disorder associated with undesirable vascular SMC proliferation and/or inhibiting neointima formation, the peptide comprising, consisting essentially of, or consisting of the amino acid sequence LDPAKDCGDQKYAY (SEQ ID NO: 1).

16. The peptide for use of claim 15, wherein the peptide comprises an N-terminal stearate modification.

17. A method for identifying an inhibitor of a cyclin E/connexin43 interaction, the method comprising:
(a) combining a cyclin E polypeptide and a connexin43 polypeptide in the presence of the peptide of claim 1 under conditions sufficient to induce formation of a complex between the cyclin E polypeptide and the connexin43 polypeptide; and
(b) determining an extent of binding of the peptide of claim 1 to the complex in the presence and the absence of a candidate inhibitor of a cyclin E/connexin43 interaction,
wherein a reduction in biding of the peptide of claim 1 to the complex in the presence of the candidate inhibitor of a cyclin E/connexin43 interaction as compared to in the absence of the candidate inhibitor of a cyclin E/connexin43 interaction is indicative of the candidate inhibitor of a cyclin E/connexin43 interaction being an inhibitor of a cyclin E/connexin43 interaction.

18. The method of claim 17, wherein the determining step comprises employing a Förster resonance energy transfer (FRET) fluorescence based method.

19. The method of claim 17, wherein the candidate inhibitor of a cyclin E/connexin43 interaction is a small molecule or an antibody or an antigen-binding fragment thereof.

20. Use of a peptide of claim 1 for identifying an inhibitor of a cyclin E/connexin43 interaction, wherein the peptide of claim 1 is employed in a competition assay with one or more candidate inhibitors of a cyclin E/connexin43 interaction.

21. The use of claim 20, wherein the identifying employs a Förster resonance energy transfer (FRET) based method.

22. The use of claim 20, wherein the one or more candidate inhibitors of the cyclin E/connexin43 interaction are small molecules or antibodies or antigen-binding fragments thereof.
